(19) United States
(12) Patent Application Publication
(10) Pub. No.: US 2024/0148991 A1
(43) Pub. Date: May 9, 2024

(54) LIQUID DISPENSER FOR NASAL APPLICATION AND PUMP DEVICE

(71) Applicant: Aptar Radolfzell GmbH, Radolfzell (DE)

(72) Inventors: Jürgen GREINER-PERTH, Gottmadingen (DE); Stefan RITSCHE, Eigeltingen (DE)

(21) Appl. No.: 18/504,384

(22) Filed: Nov. 8, 2023

(30) Foreign Application Priority Data
Nov. 9, 2022 (EP) 22206494.1

(51) Int. Cl. A61M 15/08; A61M 11/00; A61M 15/00

(52) U.S. Cl. CPC A61M 15/08; A61M 11/007; A61M 15/0015

(57) ABSTRACT

What is proposed is a nasal dispenser (10) for the nasal application of liquids in atomized form, in particular for children.

The nasal dispenser (10) has a nasal applicator (40) which is for dispensing the liquid and is formed by an outer component and an inner component, which together define a vortex chamber and at least one inlet channel into the vortex chamber.

In the case of components that are stationary in relation to one another, especially tapered inlet channels (96) for generating a high liquid velocity in the vortex chamber (98) are proposed. The cross-sectional area of the inlet channel (96), that is aligned transversely to the flow direction (4) in the inlet channel, at its narrowest point or the sum of the cross-sectional areas of the inlet channels, that are aligned transversely to the flow direction (4) in the inlet channels (96), at their respective narrowest points is at most 0.05 mm².

In the case of a design in which the inner component (60) is arranged movably in the outer component (50), in particular for the purpose of obtaining a valve function, it is conversely proposed to design the outlet opening (54) with a narrowest cross section of at most 0.05 mm².

### CROSS-REFERENCE TO RELATED APPLICATION

This claims priority from European Application No. 22206494.1, filed Nov. 9, 2022, the disclosure of which is hereby incorporated by reference in its entirety.

### FIELD OF APPLICATION AND PRIOR ART

The invention relates to a nasal dispenser for the nasal application of atomized liquids and to a pump device which is in particular suitable for such a nasal dispenser.

Nasal dispensers of the type in question are used to apply liquid in atomized form nasally via the nostrils of a patient, in particular pharmaceutical liquids for relieving breathing difficulties in the case of common cold symptoms. The application of the atomized liquid in the form of a conical spray jet promotes the desired distribution of the liquid in the airways.

In the case of nasal dispensers, a spray jet of atomized liquid can be produced in various ways, for example through a nozzle plate with a multiplicity of nozzle openings or by means of a vortex chamber, in which the liquid is introduced before being discharged in a rapid swirling movement, wherein an outlet opening is provided on the end face of the vortex chamber and the swirling liquid is broken down into fine droplets when it is being discharged there.

The use of nasal dispensers which discharge liquid in atomized form is common, in particular for adults. For children, however, it is often perceived to be easier to handle nose drops, also because the burst of sprayed atomized liquid sometimes feels uncomfortable for children.

### Problem and Solution

It is an object of the invention to provide a nasal dispenser for the nasal application of liquid in atomized form, the discharge of which does not feel comfortable for children.

To achieve this object, what is proposed is a nasal dispenser for the nasal application of liquids, in particular for use with children, which is of the common type in question, having a liquid reservoir for receiving liquid before it is discharged and a slender nasal applicator for insertion into the nose and for discharging the liquid in a discharge direction. The nasal applicator is preferably part of a discharge unit that can be displaced with respect to the liquid dispenser linearly in an actuation direction and in particular parallel to the discharge direction and, by being pressed down counter to the force of a return spring, causes a pump device to be actuated or a valve device to open.

The nasal applicator has an outer component and an inner component. The outer component forms an external outer surface which is visible to the user. The outer component has a slender applicator tip with a distal end wall, through which passes an outlet opening designed to discharge a spray jet in a discharge direction. In this case, the discharge direction preferably corresponds to the main direction of extent of the slender applicator tip.

The slender applicator tip preferably has a rotationally symmetrical form, this meaning the basic shape. A design in which a thread or otherwise subordinate holding structures for fastening a cap that in all other respects, however, have a rotationally symmetrical shape are provided on the outer side is to be understood as rotationally symmetrical within the meaning of the invention. The slender shape is distinguished in that its length from a root region to the distal end is preferably 2 times, in particular at least 2.5 times, the outside diameter in the root region. Here, the root region is formed by any point which is no longer inserted upon insertion into the nostril. The length of the applicator tip from the root region to the distal end is preferably at least 15 mm. It is thus in particular preferred if the applicator tip has a diameter of at most 7.5 mm, preferably at most 6 mm, at a spacing of 15 mm from the distal end.

The inner component is inserted in the outer component. Together with the outer component, it delimits a liquid path leading to the outlet opening, in particular a vortex chamber provided in this liquid path upstream of the outlet opening. Preferably, the vortex chamber is at least partially and in particular predominantly formed by a depression in the inner component. The inner component may additionally form an outlet channel which connects the vortex chamber to a pump device. In other configurations, the outlet channel is arranged between the inner component and the outer component.

At least one swirl-imparting inlet channel leads into the vortex chamber, wherein preferably there are provided at least two such inlet channels that are opposite one another, or more than two inlet channels leading into the preferably rotationally symmetrical vortex chamber in circumferentially distributed fashion. The inlet channels are angled with respect to a radial direction in order to allow the liquid to flow into the vortex chamber in such a way that it attains a rotating swirling movement there.

The inlet channel or the inlet channels are preferably formed by depressions in the inner component or by apertures in an inwardly facing web of the outer component. In the event of inlet channels formed by depressions in the inner component, it is preferred if these depressions have a smaller depth than the adjacent vortex chamber, likewise formed by a depression, in order to cause a flow separation by virtue of the step that exists owing to the different depth when the liquid is flowing into the vortex chamber.

To obtain a spray jet which does not feel uncomfortable for children, it has proven to be advantageous to reduce the flow of liquid, that is to say to reduce the amount of liquid per unit of time, that is discharged through the outlet opening. Preferably, the nasal dispenser is configured such that, upon routine actuation, a flow of liquid of less than 0.7 ml/second, preferably less than 0.6 ml/second, is obtained. In the case of common nasal dispensers for adults, by contrast, a flow of liquid of 1.0 ml/second or even 1.5 ml/second is routine. In spite of the reduced flow of liquid, the components of the nasal dispenser must be designed such that the desired atomization takes place.

Purely reducing the flow of liquid, for example by way of a throttle in the outlet channel upstream of the vortex chamber and the inlet channels, has proven to not be ideal, since the reduced pressure downstream of the throttle makes it harder for the vortex chamber to form a fine atomization.

Two alternatives for obtaining a reduced flow of liquid in a manner suitable for children combined with good atomization are proposed.

A first alternative relates to a nasal applicator of which the inner component and outer component are arranged in positionally fixed fashion in relation to one another, for example in that they are latched to one another fixedly by means of a snap-fit connection. Together, they define the vortex chamber and circumferentially delimit the inlet channels into the vortex chamber.

In such a configuration, it is proposed according to the invention that the inlet channels or the at least one inlet channel have a particularly narrow design. The cross-sectional area of the inlet channel, that is aligned transversely to the flow direction in the inlet channel, at its narrowest point or, in the case of a plurality of inlet channels, the sum of the cross-sectional areas of the inlet channels at their respective narrowest points is only 0.05 mm², preferably even less, specifically 0.04 mm² or less, according to the invention.

It was found that already such a narrowing leads to a suitable reduction in the discharged flow of liquid and to an acceleration of the liquid, which has the effect of a high degree of atomization. The very fine inlet channels thus allow reliable atomization with a reduced flow of liquid. In addition, the inlet channels are particularly suitable as the location of the narrowest point of a discharge channel, since they can be provided with the desired cross section very easily and precisely by suitable shaping of the inner component and the outer component.

The at least one inlet channel preferably has a cross section which tapers in the flow direction, with the result that the narrowest point is provided with a clear cross section, according to the invention of at most 0.05 mm², at an opening into the vortex chamber. The velocity of the liquid therefore continuously increases when it flows in the flow direction towards the vortex chamber.

The size of the outlet opening does not decisively matter in the described configuration with very fine inlet channels, since the inflow velocity of the liquid into the vortex chamber is primarily responsible for good atomization in this case. It is therefore proposed that the outlet opening has a relatively large configuration. In the sense of straightforward production, the outlet opening at its narrowest point should a cross-sectional area which is greater than the cross-sectional area of the inlet channel or than the sum of the cross-sectional areas of the inlet channels. In particular preferably, the outlet opening at its narrowest point has a cross-sectional area of at least 0.06 mm² auf, in particular of at least 0.08 mm².

In this first alternative, the inlet channels are formed around the circumference by the outer component and the inner component together. They are virtually formed by a gap which remains between an end face of the inner component and the inner side of the end wall of the outer component. In particular, depressions that form the inlet channels may be provided in an upper end face of the inner component that bears against the inner end wall of the outer component. The inlet channels are formed by mutually opposite channel walls of the two components, i.e. of the outer component and of the inner component. The spacing between these channel walls defines the channel height. Preferably, the channel height is less than 0.15 mm, preferably less than 0.12 mm. This narrow gap can be created reliably and with low manufacturing outlay in that the mentioned depressions of corresponding depth are provided on the end face of the inner component.

The inlet channels are preferably provided with a greater width than they are height. In particular preferably, the channel width, that is to say the extent of the cross-sectional area of the respective inlet channel at its narrowest point, transversely to the flow direction and transversely to the channel height, is twice the channel height. The channel width is preferably at least 0.15 mm, in particular preferably 0.18 mm or more.

The second alternative proposed here relates to a nasal applicator in the case of which the inner component is arranged movably in the outer component. In particular, the inner component is arranged so as to be linearly displaceable in the outer component, wherein it is pressed away from the outlet opening counter to the force of a return spring in particular by an increase in the pressure of the liquid.

In such a configuration with a displaceable inner component, it is difficult to design the inlet channels in such a way that they generate a reproducible throttle action and thereby a reduced flow of liquid and reliably atomize the liquid, since in such a configuration the inner component and the outer component together delimit the at least one inlet channel and the minimum cross section of the at least one inlet channel is variable owing to the movability.

In such a case with a displaceable inner component, it is proposed according to the invention to give the outlet opening a particular shape in order to omit a throttle action and at the same time obtain a sufficient degree of atomization. To this end, the outlet opening is designed with a particularly small cross section, specifically with a clear cross-sectional area at its narrowest point of at most 0.05 mm², preferably at most 0.04 mm². The outlet opening may in particular have a rotationally symmetrical shape.

It is therefore provided that the atomization is not influenced primarily by influencing the velocity as the liquid flows into the vortex chamber, but rather by a particularly small outlet opening. It has been shown that this also enables the atomization and the generation of a conical spray jet with a small flow of liquid. In particular, this can be achieved if the ratio of an average diameter of the outlet opening to a maximum diameter of the pump chamber is between 1:3 and 1:5.

The inlet channels may in particular be provided in a web which surrounds the inside of the outlet opening. Depending on the relative position of the inner component, it may partially close the inlet channels in the manner of an internal stop and thus cause the variable cross section mentioned.

The inner component and the outer component preferably together form a discharge valve. In this case, the inner component has a closing piston, which in the end position covers the at least one inlet channel in the web on the inside and as a result completely prevents liquid from flowing from the inlet channel into the vortex chamber in an end position. As an alternative or in addition, it may be provided that the inner component and the outer component axially seal one another off in an end position in that an end face of the inner component bears against an inner side of the end wall of the outer component on the inside.

The production of a small outlet opening proposed according to the invention, which passes through a comparatively large outer component, can be facilitated in that the outlet opening has a cross section which is non-uniform over the thickness of the end wall, that is to say does not have the small cross section mentioned over the entire thickness of the end wall. Preferably, on the inner side or on the outer side of the end wall, that is to say on the inlet side or outlet side, the outlet opening has an in particular conical or cylindrical depression which is virtually part of the outlet opening or constitutes a tapering of the end wall. This depression makes it easier to achieve the desired precision of the outlet opening in the case of an outer component produced by means of plastics injection moulding.

A nasal dispenser according to the invention discharges pressurized liquid. One possible design provides that the liquid is stored in a liquid reservoir in the form of a pressurized reservoir and is released by a manually actuated valve. It is preferable, however, if the liquid is not stored under pressure in the liquid reservoir and is conveyed to the outlet opening by means of a pump device.

In particular preferably, a pump device, which is connected to the liquid reservoir by way of an inlet side and is connected to the outlet opening via an outlet channel, is thus provided. To actuate the pump device, an actuator which is displaceable between an unactuated end position and an actuated end position is provided, wherein this actuator is preferably provided positionally fixed in relation to the applicator tip and, together with the applicator tip, is pressed down with respect to the liquid reservoir in order to convey liquid from the pump chamber of the pump device to the outlet opening.

The pump device is preferably a piston pump with a cylinder wall and a piston which provides sealing in relation thereto and which can be used to vary a pump chamber volume. On the inlet side and outlet side, the pump device has an inlet valve and an outlet valve.

The outlet valve is preferably in the form of a pressure-dependently opening outlet valve which opens when there is excess pressure in the pump chamber. In a configuration in which the inner component and the outer component are movable in relation to one another and together form an outlet valve, this outlet valve may at the same time be the outlet valve of the pump device or be provided as additional valve in addition to a separate outlet valve of the pump device.

The inlet valve may likewise be a pressure-dependently opening valve which opens when there is excess pressure in the pump chamber and as a result takes in liquid from the liquid reservoir, in particular through a riser pipe. A directionally dependently opening inlet valve, however, is preferred over a pressure-dependently opening inlet valve. This is yet to be described below.

The pump device is preferably designed to convey a relatively small amount of liquid per actuation, in order not to make the discharge feel uncomfortable for a child. It is preferred if the nasal dispenser rather is used multiple times in succession for application into the same nostril of the child, instead of completely discharging the desired amount of liquid with one actuation.

In particular, the pump device may be designed to convey an amount of liquid of less than 70 μl, in particular preferably less than 50 μl or even less than 30 μl, to the outlet opening when the actuator is displaced from the unactuated end position to the actuated end position.

In this case, it is in particular preferred, although not compulsory, for this small amount of liquid to be conveyed in the course of an actuation from the unactuated end position to the actuated end position of at least 4 mm, in particular at least 6 mm, wherein the actual conveying operation only needs to take place during one segment of this actuation. The relatively large actuation length facilitates operation and lowers the necessary actuation force.

As already explained in the introduction, it is preferred if the nasal dispenser is designed for a relatively small flow of liquid, that is to say in particular for achieving a discharge of less than 0.7 ml/second with an average flow of liquid. In a configuration with a pump device, the pump device and a discharge channel, adjoining it here, as far as the outlet opening are therefore preferably configured in such a way that, upon actuation with a reference force of 25 newtons, the actuation from the unactuated end position to the actuated end position leads to an average flow of liquid of less than 0.7 ml/second, preferably to a flow of liquid of less than 0.5 ml/second or less than 0.3 ml/second. Here, only that period of time in which discharge takes place, that is to say when the outlet valve of the pump device is open, is taken into account.

In addition to the described nasal dispenser, the invention also relates to a pump device for a liquid dispenser, in particular a pump device for use in a nasal dispenser of the type described above. A pump device suitable for dispensing a small and precisely metered amount of liquid is particularly preferred for a nasal dispenser of the type described.

To achieve this, the pump device according to the invention has a pump chamber formed by two pump components that can be moved relative to one another, in particular a first pump component which is part of the discharge unit and a second pump component which is part of a base positionally fixed in relation to the liquid reservoir. A displacement of the pump components leads to a change in the volume of the pump chamber.

The pump device has an inlet valve and an outlet valve, which connect the pump device to a liquid reservoir or an outlet opening. The outlet valve is preferably in the form of a pressure-dependently opening outlet valve and may be identical to an outlet valve of the dispenser, which may be formed by the described inner component and the described outer component.

The inlet valve of a pump device according to the invention, by contrast, is in the form of a directionally dependently opening inlet valve. This is to be understood to mean that the inlet valve is opened and closed in at least one defined relative position of the pump components. To this end, the inlet valve has a valve piston on the first pump component and a metering channel on the second pump component. Upon actuation from the starting position to the end position, the valve piston sealingly enters the metering channel to close the inlet valve and thus to bring about the discharge. The valve piston thus sealingly bears against the inner side of the metering channel. Preferably, the valve piston and the metering channel are dimensioned such that the valve piston can disengage from the metering channel again by way of its sealing surface at the opposite end and thus a sudden end to the discharge towards the end of the actuation is achieved.

Directionally dependently opening inlet valves are in principle already known, but known valves of this type are designed for greater flows of liquid. In order to design a pump device according to the invention of the type described above with a metering channel and a valve piston with a small conveying volume, it is considered preferable according to the invention if the pump device has a cylindrical pump chamber wall on the first pump component and a piston surface, bearing against the inner side of the cylindrical pump chamber wall, on the second pump component, this being achieved in structural terms in such a way that a sleeve that forms both the metering channel and the piston surface is provided on the second pump component. The sleeve points into an inner region of the cylindrical pump chamber wall. The inner side of the sleeve forms the metering channel. The piston surface that permanently bears against the cylindrical pump chamber wall during operation is provided on the outer side of the sleeve and/or at the distal end of the sleeve.

This design makes it possible to configure the pump chamber with a very small cross section, since the described sleeve performs a dual function. Since it is a direct support for the piston surface, the cylindrical pump chamber wall can be configured with an only slightly larger cross section than the metering channel. Correspondingly, the conveying volume is comparatively small in relation to the actuation travel.

In particular, it may be provided that the cross-sectional area of the cylindrical pump chamber wall is between 10 mm² and 20 mm², preferably between 10 mm² and 17 mm². The cross-sectional area of the metering channel is preferably between 2 mm² and 5 mm², in particular between 3 mm² and 4 mm². The effective piston surface area of the pump chamber for conveying the liquid is preferably between 6 mm² and 15 mm². In the case of an effective piston surface area of 10 mm², by way of example, an amount of liquid of 50 μl is discharged over a metering travel of 5 mm.

The invention relates to a nasal dispenser of the type described both in the unfilled state before it is filled and in the finished state for the end customer with a filled liquid reservoir. In the filled state, the liquid reservoir is preferably filled with a total amount of liquid of less than 50 ml, preferably less than 30 ml.

In the filled state, a pharmaceutical liquid is present in the liquid dispenser, this also being understood to mean in particular saline aqueous solutions. In particular, the pharmaceutical liquid may also be a liquid with a decongesting action, in particular a liquid with an imidazoline constituent, such as oxymetazoline.

### BRIEF DESCRIPTION OF THE DRAWINGS

Further advantages and aspects of the invention will emerge from the claims and from the following description of preferred exemplary embodiments of the invention, which are explained below on the basis of the figures.

FIGS. 1 and 2 show a first exemplary embodiment of a nasal dispenser according to the invention in an unsectioned and a sectioned illustration.

FIGS. 3 to 5 show the outlet opening and the vortex chamber, upstream of the outlet opening, of the nasal dispenser of FIGS. 1 and 2.

FIGS. 6 and 7 show a second exemplary embodiment of a nasal dispenser according to the invention in an unsectioned and a sectioned illustration.

FIGS. 8 to 10 show the outlet opening and the vortex chamber, upstream of the outlet opening, of the nasal dispenser of FIGS. 6 and 7.

### DETAILED DESCRIPTION OF THE EXEMPLARY EMBODIMENTS

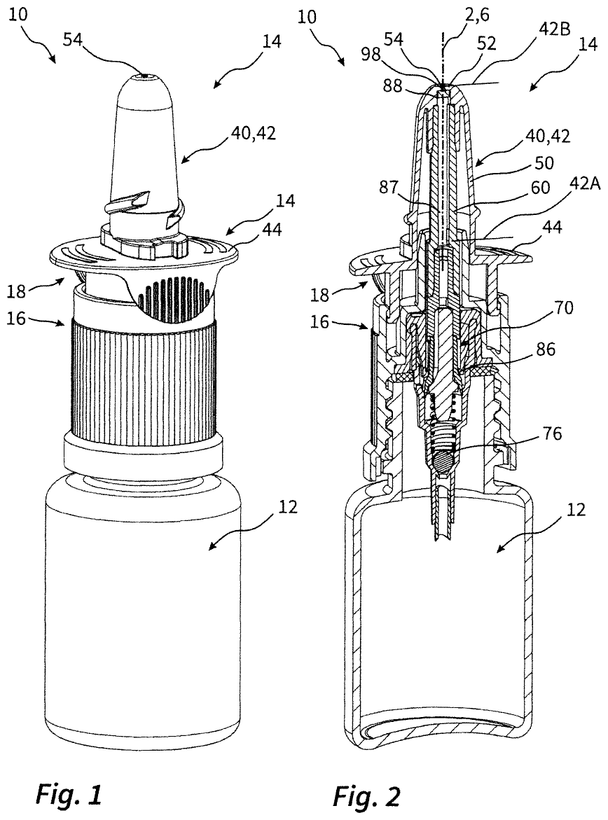

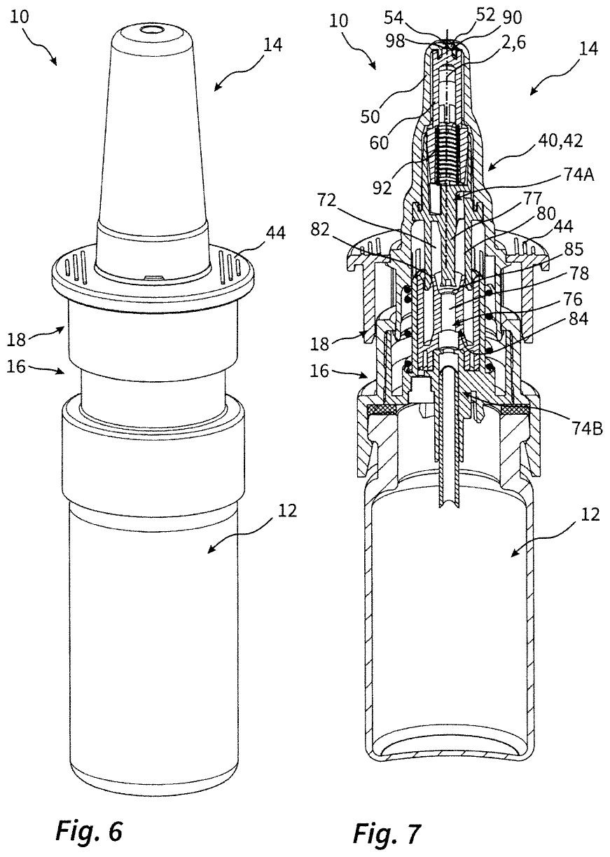

FIGS. 1 to 5 depict a first exemplary embodiment of a nasal dispenser 10 according to the invention. FIGS. 6 to 10 depict a second exemplary embodiment of a nasal dispenser 10 according to the invention.

It holds true for both nasal dispensers 10 that they have a liquid reservoir 12, on which a discharge head 14 is mounted. This discharge head 14 has a base 16, which is connected to the liquid reservoir for example by means of a threaded or a snap-fit connection.

The discharge head 14 also has a discharge unit 18, which is mounted movably on the base 16 and can be pressed down towards the base 16 in an actuation direction 6. The discharge unit 18 has a slender nasal applicator 40 in the form of an applicator tip 42, which tapers towards a distal end and is intended to be pushed into a nostril of a user. The discharge unit also has an actuator 44 in the form of a finger support running around the circumference.

An outlet opening 54, which is upstream of a vortex chamber 98, is provided at the distal end of the applicator tip 42. The outlet opening 54 is in the form of an aperture in the end of an outer component 50. The vortex chamber 98 is delimited by the inner side of an end wall 52 of this outer component and by an inner component 60.

The nasal dispensers each have a pump device 70, which can be actuated by pressing down on the discharge unit 18 with respect to the liquid reservoir 12 and the base 16. The pump device conveys liquid out of the liquid reservoir 12 to the outlet opening 54, and the liquid flows into the vortex chamber 98 through inlet channels 96. As a result of the alignment of the inlet channels 96, which is angled with respect to a radial direction, the liquid is made to swirl when it enters the vortex chamber 98. The liquid that has entered the vortex chamber 98 therefore rotates clockwise or anticlockwise at high velocity.

The rotating liquid arrives at the end wall 52 and the outlet opening 54 provided therein. The swirl in the liquid has the effect that the liquid exiting through the outlet opening 54 is broken down into a conical spray jet of fine individual droplets when its velocity is high enough.

The nasal dispensers 10 illustrated in FIGS. 1 to 5 and 6 to 10 constitute two configurations of nasal dispensers 10 which differ in terms of essential details and are intended in particular for use with children. The nasal dispensers 10 have the shared feature that they are specifically designed to generate a discharged flow of liquid of only approximately 0.7 ml/second. This small flow of liquid, together with fine atomization, ensures that the discharge does not feel very uncomfortable to children. Depending on the extent to which the flow of liquid is reduced, it is even possible to have the effect that the discharge is barely perceived by children at all.

In the case of the configuration in FIGS. 1 to 5, it is provided that the discharge unit 18 and the nasal applicator 40 provided thereon is formed by essentially two components, specifically the outer component 50 and the inner component 60. The liquid is conveyed to the outlet opening 54 by a pump device 70. The pump device 70 has an inlet valve 76 and an outlet valve 86. On the far side of the outlet valve 86, the liquid flows towards the outlet opening 54 through a channel 87, a partial portion of which is surrounded by the inner component 60. At the end of this channel 87, the liquid enters the channel portion 88, which is at the end and is delimited by the inner component 60 and the outer component 50 together, through radial openings. The liquid arrives at two inlet channels 96 through this channel portion 88.

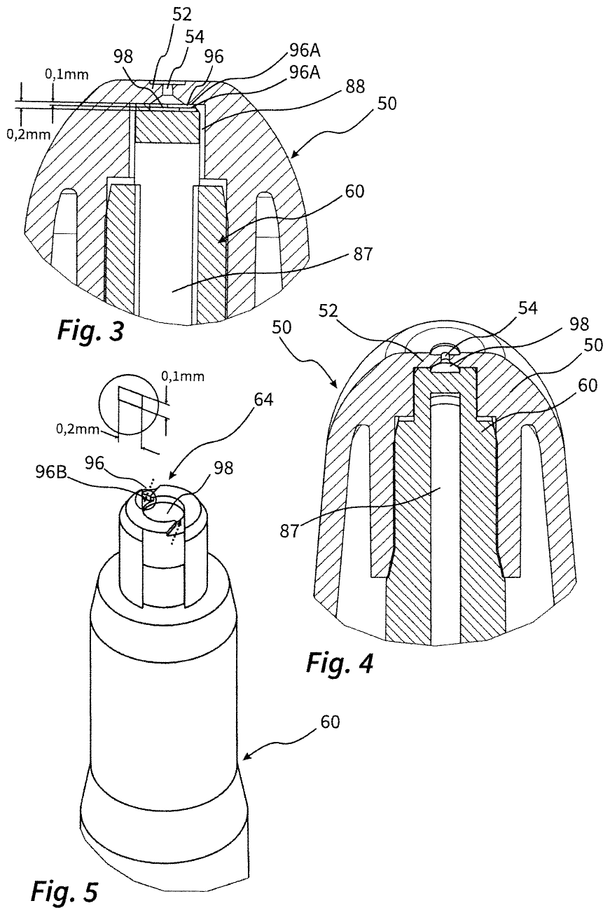

The inlet channels 96, as can be seen in particular in FIG. 3, are delimited by mutually opposite channel walls 96A, 96B, wherein the upper channel wall 96A shown in FIG. 3 is formed by the inner side of the end wall 52 of the outer component 50 and wherein the lower channel wall 96B shown in FIG. 3 is formed by the end face 64 of the inner component 60.

The end face 64 of the inner component 60 can be seen in particular in FIG. 5. The end face is formed with a central depression having a depth of for example 0.2 mm, which together with the end wall 52 forms the vortex chamber 98. The inlet channels 96 are decisively formed by two further depressions having a smaller depth of for example 0.1 mm, with the result that liquid flowing in through them flows into the vortex chamber 98 over a step and as a result a relatively clean flow separation is caused.

The inlet channels have a tapering shape with respect to a flow direction 4. At their narrowest cross section, they face into the vortex chamber 98 directly upstream of the opening. There, a width of the inlet channels transversely to the flow direction 4 is 0.2 mm. The overall cross section of each of the two inlet channels is therefore 0.02 mm², in total 0.04 mm², as can be seen from the enlarged region illustrated in FIG. 5.

The outlet opening 54 is larger than the sum of the cross sections of the inlet channels, and therefore the inlet channels constitute the overall narrowest point between the pump device 70 and the outlet of the liquid. Preferably, the outlet opening is shaped in rotationally symmetrical fashion and at its narrowest point has a diameter of at least 0.25 mm and preferably at least 0.3 mm, that is to say a smallest clear cross section of at least approximately 0.05 mm² and preferably at least approximately 0.1 mm².

The very narrow inlet channels 96, in combination with the step at the transition between the inlet channels 96 and the vortex chamber 98, cause a largely disruption-free flow of the liquid at high velocity. At the same time, the narrow inlet channels 96 reduce the flow of liquid that is achieved with a routine actuation velocity, preferably to approximately 0.3 ml/second. In spite of this reduced flow of liquid, the high velocity of the liquid as it flows into the vortex chamber 98 brings about very good atomization. The diameter of the outlet opening 54 therefore scarcely matters. In the case of the embodiment illustrated here, the diameter of the outlet opening 54 is approximately 0.3 mm. However, larger diameters would also be possible, since the advantageous atomization is primarily ensured by the inlet channels 96 in this configuration.

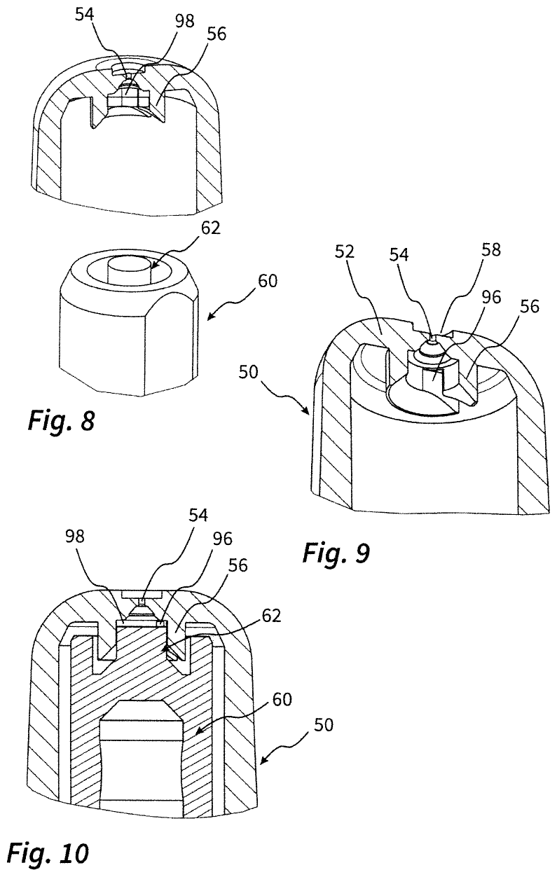

In the case of the configuration of FIGS. 6 to 10, it is provided that the outer component 50 and the inner component 60 are movable relative to one another and together form an outlet valve 90. As can be seen with reference to FIGS. 8 to 10 and in particular with reference to FIG. 9, a web 56, which runs around the circumference and is interrupted by slits that together with the inner component 60 form the inlet channels 96, is provided on the inner side of the end wall 52. By way of its distal end, the inner component 60 forms a closing piston 62, which is movable with respect to the web 56. If there is no liquid pressure, the outlet valve 90 is closed. The closing piston in this state is engaged with the web 56 to a great enough extent that the slits there are completely covered and thus no liquid can flow into the vortex chamber 98 delimited by the web and the closing piston. If pressure is applied to the liquid by means of the pump device 70 of the nasal dispenser 10, the liquid pressure presses the inner component 60 away from the outlet opening 54 counter to the force of a valve spring 92, with the result that the vortex chamber 98 is enlarged and access for the liquid through the inlet channels 96 is opened up.

Since the inlet channels are not readily suitable as narrowing for achieving a reproducible throttle action owing to the pressure dependency of the opening of these inlet channels, in the configuration of FIGS. 6 to 10 the outlet opening 54 conversely has a very small configuration. It has been shown that it is also possible to achieve a small flow of liquid and the desired atomization as a result of this.

The outlet opening 54 at its narrowest point therefore has a cross-sectional area of 0.04 mm² or less. In particular preferably, the outlet opening has a rotationally symmetrical form and has a smallest diameter of 0.2 mm and correspondingly a cross-sectional area of less than 0.035 mm².

To make it easier to produce such a fine outlet opening at the narrowest point, it is provided that this end wall 52 has depressions 58 on the inner side and/or the outer side in the region of the outlet opening. The maximum thickness of the end wall of approximately 0.5 mm to 0.8 mm is thus reduced in the region of the outlet opening by virtue of the depressions 58, preferably to a thickness between 0.2 mm and 0.4 mm. This thickness of preferably between 0.2 mm and 0.4 mm is also preferably passed through by a cylindrical portion of the outlet opening 54, which continuously has the small cross-sectional area mentioned of less than 0.04 mm², preferably less than 0.035 mm².

The vortex chamber 98 preferably has a maximum diameter which is approximately four times to eight times the diameter of the outlet opening 54 and therefore in particular preferably is between 0.8 mm and 2.0 mm, in particular between 0.8 mm and 1.2 mm. The vortex chamber 98 preferably transitions into the cylindrical portion mentioned of the outlet opening 54 via a conically narrowed region, in particular formed by the depression mentioned on the inner side.

When the nasal dispenser 10 is actuated by pressing down on the discharge unit 18, the inlet channels 96 open in the manner depicted in FIG. 10 as a result of pressure, since the closing piston 62 is spaced apart from the outlet opening. The liquid flows into the vortex chamber 98 and is discharged through the very narrow discharge opening 54. Although the inlet channels, depending on the position of the closing piston, are significantly larger than in the configuration of FIGS. 1 to 5, a considerable actuation resistance and the desired atomization together with a small flow of liquid of less than 0.3 ml/second is achieved owing to the narrow outlet opening 54.

Apart from the configuration of the nasal dispenser 10 of FIGS. 6 to 10 in terms of the vortex chamber 8 and the outlet opening 54, the pump device 70 is configured in a particular way. The pump device has an inlet valve 76 and an outlet valve 90. The outlet valve 90 is identical to the already mentioned outlet valve 90. However, configurations with two independent valves downstream of the pump chamber 72 of the pump device 70 are also possible.

As a departure from the configuration of FIGS. 1 to 5, the inlet valve 76 does not have a pressure-dependently opening inlet valve but a directionally dependently opening one.

It comprises a first pump component 74A, which is part of the displaceable discharge unit 18 and, including the inner component 60, is fixedly connected to the outer component 50. This first pump component 74A has a central valve piston 77 and a web which surrounds this valve piston 77 around the circumference and the inner side of which forms a pump chamber wall 80.

A second pump component 74B is part of the base 16. This second pump component 74B has a central sleeve 84, which performs the following functions: An inlet channel 85 passes through the sleeve 84, wherein a portion of the inlet channel 85 of small cross section forms a metering channel 78. This metering channel is designed such that the valve piston 77 of the first pump chamber component closes the inlet channel 85 when arranged in the metering channel and interrupts the communicating connection between the pump chamber 72 and the liquid reservoir 12.

The sleeve 84 is furthermore a support for a piston surface 82, which is provided at a distal end of the sleeve and defines the maximum sleeve diameter. The piston surface 82 bears against the pump chamber wall 80.

If the discharge unit 18 is now pressed down, first of all the liquid is pressed back out of the pump chamber 72 to the liquid reservoir 20, through the still-open inlet channel 85, until the valve piston 77 enters the metering channel 78. The backflow is then interrupted, and continuing to press down on the discharge unit 18 causes the pressure to rise in the pump chamber 72 and correspondingly the outlet and discharge valve 90 to open. This discharge takes place over an actuation travel of 5 mm, this being a consequence of the length of the metering channel 78. As soon as the valve piston 77 exits the metering channel 78 at the lower end, the communicating connection between the pump chamber 72 and the liquid reservoir 12 is restored. The excess pressure in the pump chamber is discharged in the liquid reservoir 12 and the discharge and outlet valve 90 closes.

During the discharge, that is to say during the period of time between the closing of the inlet valve 76 and the opening of the inlet valve 76, the liquid is conveyed out of the pump chamber 72 towards the outlet opening 54.

Since the sleeve 84 performs a dual function and itself supports the piston surface 82, a pump chamber with a very small diameter can be obtained. In the present case, the diameter of the cylindrical pump chamber wall is 4 mm and the cross-sectional surface area is thus approximately 12.5 mm². The effective piston surface area, taking into account the valve piston 77, is somewhat smaller still and is approximately 7 mm² to 10 mm², in the present case is 8 mm². Accordingly, 40 μl of liquid is conveyed and discharged over the metering travel of 5 mm.

The geometry of the liquid path from the pump chamber 72 to the outlet opening 54 has the effect that the discharge is effected at a reference actuation force of 25 newtons over a period of time of approximately 0.15 seconds, that is to say with an average flow of liquid of 0.27 ml/second. This leads to a gentle spray jet which is virtually imperceptible in the nose and does not feel very uncomfortable to children.

1. Nasal dispenser (10) for the nasal application of liquids in atomized form, in particular for the nasal application of liquids for children, having the following features:
a. the nasal dispenser (10) has a liquid reservoir (12) for receiving liquid before it is discharged, and
b. the nasal dispenser (10) has a nasal applicator (40) for discharging the liquid, and
c. an outer component (50) of the nasal applicator (40) has a slender applicator tip (42) with a distal end wall (52), through which passes an outlet opening (54) designed to discharge a spray jet in a discharge direction (2), and
d. an inner component (60) of the nasal applicator (40) is inserted in the outer component (50), and
e. the outer component (50) and the inner component (60) together delimit a vortex chamber (98) upstream of the outlet opening (54), wherein at least one swirl-imparting inlet channel (96) leading into the vortex chamber (98) is provided,
characterized by one of the following further features:
f. the inner component (60) and the outer component (50) are arranged in positionally fixed fashion in relation to one another and together circumferentially delimit the at least one inlet channel (96), wherein the cross-sectional area of the inlet channel (96), that is aligned transversely to the flow direction (4) in the inlet channel, at its narrowest point or the sum of the cross-sectional areas of the inlet channels, that are aligned transversely to the flow direction (4) in the inlet channels (96), at their respective narrowest points is at most 0.05 mm², preferably at most 0.04 mm², or
g. the inner component (60) is arranged movably in the outer component (50) and the inner component (60) and the outer component (50) together delimit the at least one inlet channel (96), with the result that the cross section of the at least one inlet channel (96) can be varied by virtue of the movability of the inner component (60) with respect to the outer component (50), and the outlet opening (54) at its narrowest point has a cross-sectional area of at most 0.05 mm², preferably at most 0.04 mm².

2. Nasal dispenser (10) according to claim 1, having the following further features:
a. the inner component (60) and the outer component (50) are arranged in positionally fixed fashion in relation to one another, and
b. the outlet opening (54) at its narrowest point has a cross-sectional area which is greater than the cross-sectional area of the inlet channel (96) or than the sum of the cross-sectional areas of the inlet channels (96).

3. Nasal dispenser (10) according to claim 2, having the following further features:
a. the outlet opening (54) at its narrowest point has a cross-sectional area of at least 0.06 mm²,
preferably having the following additional feature:
b. the outlet opening (54) at its narrowest point has a cross-sectional area of at least 0.08 mm².

4. Nasal dispenser (10) according to one of claims 1 to 3, having the following further features:
a. the inner component (60) and the outer component (50) are arranged in positionally fixed fashion in relation to one another, and
b. the at least one inlet channel (96) at its narrowest point is designed in such a way that two opposite channel walls (96A, 96B) of the inlet channel (96), one of which is formed by the inner component (60) and one of which is formed by the outer component (50), define a channel height of less than 0.15 mm, preferably less than 0.12 mm,
preferably having the following additional feature:
c. transversely in relation to the channel height, the at least one inlet channel (96) at the narrowest cross section has a channel width which is greater than the channel height and preferably is at least twice as wide as the channel height, wherein the channel width is preferably at least 0.15 mm, in particular preferably 0.18 mm or more.

5. Nasal dispenser (10) according to one of claims 1 to 4, having the following further features:
a. the inner component (60) and the outer component (50) are arranged in positionally fixed fashion in relation to one another, and
b. the vortex chamber (98) is formed by a depression in the inner component (60), wherein the at least one inlet channel (96) is formed by a further depression in the inner component (60) with a smaller depth.

6. Nasal dispenser (10) according to claim 1, having the following further features:
a. the inner component (60) is arranged movably in the outer component (50), and
b. the inner component (60) and the outer component (50) can be displaced into an end position in which an inflow into the vortex chamber (98) is prevented,
preferably having the following additional feature:
c. the inner component (60) and the outer component (50) together form a discharge valve that can be switched by means of liquid pressure.

7. Nasal dispenser (10) according to claim 1 or 6, having the following further features:
a. the inner component (60) is arranged movably in the outer component (50), and
b. the outer component (50) has, on the inner side of the end wall (52), a web (56) which surrounds the outlet opening (54) and through which at least one inlet channel (96) passes, and
c. the inner component (60) has a closing piston (62), which in the end position covers the at least one inlet channel (96) on the inside and as a result prevents liquid from flowing from the inlet channel (96) into the vortex chamber (98) and/or in the end position bears against the inner side of the end wall (52) of the outer component (50) by way of an end face (64).

8. Nasal dispenser (10) according to claim 1, 6 or 7, having the following further features:
a. the inner component (60) is arranged movably in the outer component (50), and
b. the outlet opening (54) has a cross section which varies in the discharge direction (2) from an inner side of the end wall (52) to an outer side of the end wall (52), wherein a depression with a cross-sectional area which is greater than the narrowest point of the outlet opening (54), in particular a cylindrical and/or a conical depression (58), is formed on the inner side and/or the outer side.

9. Nasal dispenser (10) according to one of the preceding claims, having the following further feature:
a. the applicator tip (42) has a rotationally symmetrical form and tapers towards the distal end, wherein its length from a root region to the distal end is at least twice the outside diameter in the root region,
preferably having the following additional feature:
b. the length of the applicator tip (42) from the root region (42A) to the distal end (42B) is at least 15 mm.

10. Nasal dispenser (10) according to one of the preceding claims, having the following further features:
a. the nasal dispenser (10) has a pump device (70), which is connected to the liquid reservoir (12) by way of an inlet side and is connected to the outlet opening (54) via an outlet channel, and
b. an actuator (44) which can be displaced between an unactuated end position and an actuated end position is provided for actuating the pump device (70),
c. the pump device (70) is designed such that, when the actuator (44) is displaced from the unactuated end position to the actuated end position, an amount of liquid of less than 70 μl is conveyed to the at least one outlet opening (54),
preferably having at least one of the following features:
d. the pump device (70) is designed such that, when the actuator (44) is displaced from the unactuated end position to the actuated end position, an amount of liquid of less than 50 μl is conveyed to the at least one outlet opening (54), in particular less than 30 μl, and/or
e. the unactuated end position and the actuated end position are spaced apart from one another by at least 4 mm, preferably at least 6 mm, and/or
f. the pump device (70) is in the form of a plunger pump with a pump cylinder and a pump plunger, which can be displaced therein to reduce a pump chamber volume, and/or
g. the pump device (70) is designed according to either of claims 14 and 15.

11. Nasal dispenser (10) according to claim 10, having the following further feature:
a. the pump device (70) and the outlet channel to the outlet opening (54) are designed such that, in the event of an actuating force of 25 newtons, the actuation from the unactuated end position to the actuated end position leads to an average flow of liquid of less than 0.7 ml/second.

12. Nasal dispenser (10) according to one of the preceding claims, having the following further feature:
a. the nasal applicator (40) is part of a discharge unit (18) which can be displaced with respect to the liquid reservoir (12) linearly in an actuation direction (6) parallel to the discharge direction (2).

13. Nasal dispenser (10) according to one of the preceding claims, having at least one of the following further features:
a. a plurality of inlet channels (96), preferably two opposite inlet channels (96), are provided, and/or
b. the vortex chamber (98) has a rotationally symmetrical inner contour, and/or
c. the vortex chamber (98) is formed by a depression in the inner component (60), wherein the at least one inlet channel (96) is preferably formed by a further depression in the inner component (60) with a smaller depth, and/or
d. the at least one inlet channel (96) has a cross section which tapers in the flow direction (4), with the result that the narrowest point is provided at an opening into the vortex chamber (98).

14. Pump device (70) for a liquid dispenser, in particular a liquid dispenser according to one of the preceding claims, having the following features:
a. the pump device (70) has a pump chamber (72) formed by two pump components (74A, 74B) which are movable relative to one another, and
b. the pump device (70) has an inlet valve (76) and an outlet valve (90), which connect the pump device (70) to a liquid reservoir (12) and an outlet opening (54), and
c. the outlet valve (90) is in the form of a pressure-dependently opening outlet valve (90), and
d. the inlet valve (76) has a valve piston (77) on the first pump component (74A) and a metering channel (78) on the second pump component (74B), which the valve piston (77) enters to close the inlet valve (76) when the pump is being actuated,
characterized by the following further features:
e. the pump device (70) has a cylindrical pump chamber wall (80) on the first pump component (74A), and
f. the pump device (70) has a piston surface (82) which bears against the inner side of the cylindrical pump chamber wall (80), and
g. a sleeve (84), which projects into an inner region of the cylindrical pump chamber wall (80) and the inner side of which forms the metering channel (78) and the piston surface (82) is provided on its outer side and/or on its distal end, is provided on the second pump component (74B).

15. Pump device (70) according to claim 14, having the following further feature:
a. the cross-sectional area of the cylindrical pump chamber wall (80) is between 10 mm² and 20 mm², preferably between 10 mm² and 17 mm²,
preferably having the following additional feature:
b. the cross-sectional area of the metering channel (78) is between 2 mm² and 5 mm², preferably between 3 mm² and 4 mm².
